(19) United States
(12) Patent Application Publication
(10) Pub. No.: US 2008/0058782 A1
(43) Pub. Date: Mar. 6, 2008

(54) METHOD AND APPARATUS FOR MONITORING AND CONTROLLING DENSITY OF FRACTIONAL TISSUE TREATMENTS

(75) Inventors: George Frangineas, Fremont, CA (US); Leonard C. DeBenedictis, Palo Alto, CA (US); Kin F. Chan, San Jose, CA (US); Robert Kehl Sink, Mountain View, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/737,696

(22) Filed: Apr. 19, 2007

Related U.S. Application Data
(63) Continuation-in-part of application No. 11/468,275, filed on Aug. 29, 2006.

(51) Int. Cl. A61N 5/06; A61B 18/18

(52) U.S. Cl. 606/9; 606/33; 606/38

(57) ABSTRACT

A method and apparatus for fractional treatment of skin by irradiating the skin with electromagnetic energy is disclosed. Sources of the electromagnetic energy can include radio frequency (RF) generators, lasers, and flashlamps. The apparatus includes at least one sensor configured to detect a positional parameter, a skin characteristic, a skin response, or combinations thereof. The sensor provides feedback to a controller. The controller controls the treatment parameters, including the treatment density. By sensing the positional parameter, skin characteristic and/or skin response to a treatment, the controller automatically adjusts treatment density in real-time in response to the sensed positional parameter, skin characteristic and/or skin response.

### CROSS-REFERENCE TO RELATED APPLICATIONS

This application is a continuation-in-part of co-pending U.S. patent application Ser. No. 11/468,275, “Method and Apparatus for Monitoring and Controlling Thermally Induced Tissue Treatment,” by Kin F. Chan, George Frangineas, Leonard C. DeBenedictis, and Robert Kehl Sink, Aug. 29, 2006, from which priority is claimed and which is incorporated by reference in its entirety.

### BACKGROUND OF THE INVENTION

1. Field of the Invention

The present invention relates to a method and apparatus for providing fractional dermatological tissue treatment, and more particularly, to controlling treatment density as provided by an electromagnetic source based on measurements of positional parameters, skin responses, and/or skin characteristics.

2. Description of the Related Art

Many electromagnetic dermatological treatment systems require extensive training before physicians and nurses develop the skills to deliver energy uniformly over a target region, such as the face, neck, chest, or back. In many cases, physicians and nurses do not treat uniformly, resulting in uneven treatment, over-treatment, or under-treatment. There is a need to create more uniform photothermal and/or radio-frequency (RF) treatment, particularly for large areas.

However, not all patients respond the same way to the same level of treatment. So even if precisely the same laser energy dose is delivered to two different patients, the response of each patient can be substantially different. Within a single patient, the skin response can vary from region to region. Treatment of the forehead can respond differently than treatment of the neck, for example. When using fractional treatment methods, if uniform treatment densities are used for all patients or for all regions, then the treatment densities will typically be designed for the most sensitive patient or the most sensitive region in order to avoid undesirable side effects. Designing for the most sensitive region or patient will frequently lead to under-treatment of other regions or patients. Therefore, there is also a need to create fractional photothermal and/or RF treatments which can be modified in real time based on the type of treatment desired by the patient, the response of the patient to the treatment, and/or the region undergoing treatment. Thus, there is a need for fractional devices and methods which can control and adjust the density of the treatment during the treatment process. Such a system would have the capability to provide both treatments of uniform density and treatments where the treatment density is modified in real time based on the type of treatment desired, the patient's response to the treatment, and the region undergoing treatment.

Many medical laser systems for the treatment of dermatological skin conditions function by pressing a foot pedal to trigger the delivery of a single pulse of treatment energy. This type of treatment apparatus is slow and has a lot of repetitive motions, which can be tiring to the operator. Other laser treatment systems fire identical pulses at a constant pulse repetition rate as the user moves the handpiece across the tissue. This system requires skill and increases the risks of over- or under-treatment in the hands of an unskilled operator. Therefore, there is also a need for an approach to fractional electromagnetic treatment that provides controlled treatment density and adjusts the treatment density in real time to prevent over- and/or under-treatment. For fractional treatments, such an approach could control treatment density by adjusting the density in real time in response to at least one positional parameter, skin response and/or skin characteristic. Weckwerth U.S. Pat. No. 6,758,845 describes the use of optical measurements of regularly spaced indicia that are placed on or adjacent to the target region, but the concept is limited by the application of regularly spaced indicia that are counted to measure distance traveled by a handpiece. This requires the precise positioning of indicia to avoid errors. In addition, the visible indicia can be difficult to remove following treatment, and can leave an unsightly pattern on the skin following treatment.

Weckwerth '845 and Talpalriu U.S. Pat. No. 6,171,302 describe mechanical roller systems for tracking handpiece travel. These can be unreliable, for example, when used with gel due to a lack of friction between the mechanical roller and the skin surface. This leads to drop outs and errors in measurements of positional parameters. In addition, mechanical rollers can become rusted or gummed up so that they no longer spin easily, which makes dropouts and errors more likely. Wearing out of mechanical parts leads to similar errors.

Weckwerth '845 describes other systems that measure position of the handpiece indirectly, through the interaction with reference planes or points outside the target area, rather than measuring the target area directly. With this approach, the location of the target surface relative to the reference surface must be measured or controlled. In addition, these systems only measure one coordinate for the handpiece, which means that motion of the handpiece across the target tissue due to change in orientation of the handpiece can not be accounted for by the sensor systems. This leads to inaccuracies.

For treatment of large areas, an automatic laser control system is needed for adjusting laser treatment density in real time in response to positional parameters such as the handpiece position, velocity, and/or acceleration or in response to the laser treatment itself. To provide the most effective fractional treatment, to provide a treatment with few side effects, as well as for treatment of particular skin features such as blood vessels or pigmented lesions, an automatic laser control system is needed for adjusting treatment density in real time in response to skin characteristics and/or skin responses. Thus, there is a need for an apparatus and method for a feedback loop that increases the effectiveness of fractional treatment by controllably responding to treatment variables such as treatment speed, handpiece angle, handpiece acceleration, patient to patient variability, region to region variability within the same patient, etc. There is a need for fractional apparatus and methods which include a feedback loop that controllably responds to positional parameters, skin response and/or skin characteristics by adjusting the density of the fractional treatment (i.e., altering the density of treatment zones produced in a portion of skin) in real time.

There is also a need for an apparatus and method that enable faster and more reproducible fractional treatments, that require less training and skill by the operator and/or that controllably respond to treatment variables, positional parameters, skin response and/or skin characteristics by controlling treatment density. The apparatus and method can also increase effectiveness without increasing side effects or invasiveness, treat with lower pain and side effects, directly measure treatment efficacy and/or progress for use in a feedback loop either alone or with other inputs instead of relying primarily on accurate delivery of a predetermined treatment density or on measurement of handpiece positional parameters, monitor biological response of the skin to the treatment, monitor inherent skin characteristics and treatment variables for improved biological predictability, efficacy, and safety, and/or to permit better control of treatment density, for example for photo-dynamic therapy (PDT) treatments, laser hair removal, or fractional laser resurfacing. Fractional apparatus and methods can achieve such treatments by automatically controlling treatment density.

### SUMMARY OF THE INVENTION

In general, the present invention comprises an apparatus and a method for fractional treatment of skin using feedback from one or more sensors that are used to measure handpiece positional parameters, characteristics or features of the skin that is to be treated, and/or the skin response to thermal or ablative treatment that is caused by the delivery of electromagnetic energy in a fractional manner. The electromagnetic energy can be radio frequency (RF) or optical energy. The positional sensors, skin response sensors and skin characteristic sensors can be used separately or they can be advantageously combined to allow the fractional treatment to vary in response to a combination of one or more handpiece positional parameters, one or more skin characteristics, and/or one or more skin responses.

In one example, the invention is directed to a method for controlled fractional skin treatment, comprising manually moving a handpiece across a target region of skin; directing electromagnetic energy via the handpiece toward the target region in a manner so as to provide a fractional treatment, sensing at least one positional parameter, skin response and/or skin characteristic in the target region; and automatically adjusting treatment density of the fractional treatment in real-time in response to the sensed at least one positional parameter, skin characteristic and/or skin response. In one example, the fractional treatment is delivered while the handpiece is in motion. In another example, the fractional treatment is delivered while the handpiece is not in motion.

In another example, the invention is directed to a method for controlled fractional skin treatment comprising the steps described above, wherein the sensing step comprises sensing the at least one positional parameter, and the automatically adjusting step comprises automatically adjusting treatment density of the fractional treatment in real-time in response to the senses at least one positional parameter. In another example, the invention is directed to a method as described in the preceding sentence, wherein the order of the steps in the method comprises: 1) manually moving a handpiece across a target region of skin; 2) sensing a positional parameter of the handpiece; 3) directing electromagnetic energy via the handpiece toward the target region in a manner so as to provide a fractional treatment; and 4) automatically adjusting treatment density of the fractional treatment in real-time in response to the sensed positional parameter.

In another example, the invention is directed to the previously described method for controlled fractional skin treatment, wherein the sensing step comprises sensing the at least one skin characteristic, and the automatically adjusting step comprise automatically adjusting treatment density of the fractional treatment in real-time in response to the sensed at least one skin characteristic. In another example, the invention is directed to a method as described in the preceding sentence, wherein the order of the steps in the method comprises: 1) manually moving a handpiece across a target region of skin; 2) directing electromagnetic energy via the handpiece toward the target region in a manner so as to provide a fractional treatment; 3) sensing a skin response to the fractional treatment in the target region; and 4) automatically adjusting treatment density of the fractional treatment in real-time in response to the sensed skin response.

In another example, the invention is directed to the previously described method for controlled fractional skin treatment, wherein the sensing step comprises sensing the at least one skin response, and the automatically adjusting step comprises automatically adjusting treatment density of the fractional treatment in real-time in response to the sensed at least one skin response. In another example, the invention is directed to a method as described in the preceding sentence, wherein the order of the steps in the method comprises: 1) manually moving a handpiece across a target region of skin; 2) sensing a skin characteristic of the target region; 3) automatically determining an appropriate treatment density of a fractional treatment in real-time in response to the sensed skin characteristic; and 4) directing electromagnetic energy via the handpiece toward the target region in a manner so as to provide the fractional treatment of the appropriate treatment density.

In yet another example, the invention is directed to an apparatus for controlled fractional skin treatment comprising: at least one electromagnetic source configured to generate electromagnetic treatment energy capable of treating skin when delivered in a fractional manner; a manually movable handpiece operably coupled to the electromagnetic source, wherein the handpiece is configured to deliver the electromagnetic treatment energy in a fractional manner to a target region of skin so as to provide a fractional skin treatment; a controller operably coupled to the electromagnetic source, wherein the controller is configured to activate or adjust one or more parameters of the electromagnetic source, and a sensor operably coupled to the controller, wherein the sensor is configured to sense at least one positional parameter, skin response and/or skin characteristic in the target region; wherein positional, skin response and/or skin characteristic feedback from the sensor is used by the controller to control density of the fractional treatment by activation or adjustment of the one or more parameters of the electromagnetic source and/or handpiece.

In one example, a combination of relative and absolute handpiece positional measurements is measured to determine the positional changes of the handpiece relative to the target region of skin.

In one example, skin shrinkage is measured with a skin characteristic sensor and/or a skin response sensor. In other examples of the invention, one or more measured characteristics or features of the skin can include one or more of the following: skin birefringence, skin water content, skin elasticity, skin mechanical damping parameters, skin color, skin features such as blood vessels and pigmented lesions, skin thickness, skin texture, and wrinkles. In other examples of the invention, one or more measured responses of the skin can include changes in one or more of the following: skin birefringence, skin water content, skin elasticity, skin mechanical damping parameters, skin color, skin features such as blood vessels and pigmented lesions, skin thickness, skin texture, and wrinkles. These and other skin characteristics and/or skin responses can be measured using one or more types of technology such as capacitive sensors, (hyper-) spectral imaging, terahertz imaging, optical coherence tomography, confocal microscopy, ultrasonic imaging, coherent detection, thermal detectors, thermal imaging systems, etc. Other skin characteristics and/or skin responses and measurements can also be used.

In one example, the output of an erbium doped fiber laser is collimated and deflected by a scanning delivery unit such as a galvanometer scanner or a starburst scanner as described in the co-pending and co-owned U.S. application Ser. No. 11/158,907, which is incorporated by reference herein, to create a series of figures at the target region.

In another example, the scanning rate of the scanning delivery unit is controlled by a controller to deliver a predefined pattern or dosage even if the handpiece velocity changes within a chosen range.

In one example, a contrast enhancing agent is used to enhance the signal to noise ratio of the positional sensor. For example, Food, Drug & Cosmetic (FD&C) Blue #1 can be applied to the surface of the skin to create an improved signal for a positional sensor comprising an optical mouse chip, charge-coupled device (CCD) array, or other detector array, with, for example, at least 25 elements. Using at least 25 elements as a 5×5 array allows sufficient image resolution to observe the changes in positional parameters, skin response, skin characteristics and/or dosage response. If fewer detector elements are used, a more sophisticated algorithm and/or more sophisticated electronics generally will be typically required in order to distinguish changes in handpiece positional parameters, skin response and/or skin characteristics. Other contrast enhancing agents are fluorescent or provide maximum contrast enhancement with infra-red (IR) or ultra-violet (UV) illumination. Wavelength selective coatings on the optical elements of the system can be used in conjunction with fluorescent contrast enhancing agents to filter out one or more illumination wavelengths. For example, the wavelength selective coatings can be designed to filter out light that is used to enhance the response of an optical positional sensor in order to improve the signal to noise ratio for a fluorescent emission signal at a different wavelength.

The contrast enhancing agent can be applied as a uniform or non-uniform pattern of similar or dissimilar shapes. This pattern of contrast enhancing agent can be applied using rollers, stamps, sprays, and/or stencils, for example. The contrast enhancing agent can also be applied onto or into an adhesive substance such as used in a temporary tattoo.

In other examples, the positional sensor comprises one or more of the following: a mechanical mouse wheel or roller ball, non-concentric coils, an accelerometer, a gyroscope, transmitter(s) and receiver(s) that can be used to measure distance, a Doppler radar system, an ultrasonic time of flight measurement, etc.

In another example, leading and trailing dosage evaluation sensors are used to measure the differential skin response due to thermal treatment.

In another example, the scanning motion of a scanning delivery unit is not changed, but the pulse rate or pulse timing of the electromagnetic source is changed by the controller so as to change treatment density in response to measurements by at least one positional sensor, skin response sensor and/or skin characteristic sensor. Additionally, the pulse timing and scanner patterns can be chosen such that the beam is intentionally dragged across the target region to reduce the treatment intensity and/or to increase the size of each treatment zone created by each energy pulse.

In fractional treatment, healthy skin is spared in regions between individual treatment zones to create a treatment with a given treatment density. The spared tissue helps to promote rapid healing of the wounded area, prevent scarring, and allow higher treatment levels than are otherwise possible without side effects. The measurement of positional parameters, skin response and/or skin characteristics can be used to control the number of treatment zones created and/or to accurately space the treatment zones from one another so that treatment density can be properly controlled. The density of the fractional treatment can be controlled through the use of feedback from positional, skin response, and/or skin characteristic sensors.

Other aspects of the invention include methods, devices, and systems corresponding to the approaches described above, as well as applications of the foregoing.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 is a diagram of an example of the invention that incorporates a positional sensor and a skin sensor (i.e., a skin response sensor and/or a skin characteristic sensor).

FIGS. 2A, 2B, and 2C are diagrams of an example of the invention that incorporates an optical source, a starburst scanner wheel, and an optical positional sensor. FIG. 2C also depicts one possible treatment pattern created by this example.

FIGS. 3A, 3B, and 3C are illustrations of patterns that can be applied to the target region or to regions adjacent to the target region to enhance the measurements of the optical positional sensor shown in FIG. 1.

FIG. 4 is a diagram of an example of the invention wherein one or more accelerometers are attached to the handpiece to measure positional parameters of the handpiece in up to three dimensions and/or up to three angular orientations.

FIG. 5 is a diagram of an example of the invention wherein transmitters and receivers are used to triangulate the position of the handpiece to measure positional parameters in up to three dimensions and/or up to three angular orientations.

FIGS. 6 and 7 are diagrams of examples of the invention wherein at least one ultrasonic transmitter and at least one ultrasonic receiver are mechanically coupled to the handpiece. The example depicted in FIG. 6 utilizes an ultrasonic time-of-flight measurement. The example in FIG. 7 utilizes an ultrasonic reflection measurement.

FIG. 8 illustrates an example of the invention wherein polarized imaging is used to measure changes in the birefringence of the skin.

FIG. 9 is a diagram showing the use of leading and trailing dosage evaluation sensors in accordance with the invention for the measurement of the differential skin response to particular treatment parameters.

FIGS. 10 and 11 illustrate examples of the invention that measure the skin response to particular treatment parameters by measuring the signature of a shock wave created by an energy pulse incident on the skin. FIG. 10 illustrates an apparatus for measuring the shock wave signature with a piezo-electric material. FIG. 11 illustrates an apparatus for measuring the shock wave signature with a reflected probe beam.

FIG. 12 is a diagram of an example of the invention wherein one or more coil sensors are used to measure positional parameters of the handpiece.

FIG. 13 illustrates measurements created by a system according to FIG. 12.

### DETAILED DESCRIPTION

This invention describes an electromagnetic system with automatic adaptive control of fractional (photothermal and/or RF) treatment density, as well as a method of treating tissue in a fractional manner using an electromagnetic system with automatic adaptive control of treatment density. Treatment density is the number of treatment zones produced per unit surface area in a target region of skin or portion thereof. A nominal pattern and treatment density can be defined when the system begins treatment and this treatment density can be modified based on a measured position of the handpiece, an intrinsic characteristic of the tissue undergoing treatment and/or a change in an intrinsic characteristics of the tissue, a skin response to the treatment and/or a change in a skin response to the treatment. Sensors of various types can be used to determine the measured position parameter, skin characteristic and/or skin response. Algorithms that describe the positional parameter, skin characteristic and/or skin response to treatment are used to control the density of the fractional treatment.

As described herein, handpiece positional parameters include parameters relating to the position, velocity and/or acceleration of the handpiece used to deliver the fractional treatment. Skin characteristics include inherent characteristics, biological properties and/or features of the skin, such as, for example, skin tone, birefringence, temperature, water content, pH, elasticity, color, thickness, texture, mechanical damping properties, blood vessels, pigmented lesions, hairs, hair follicles, wrinkles, etc. Skin characteristics can include changes in these inherent characteristics, biological properties and/or features of the skin which are not brought about by or in response to the treatment. Skin responses include changes in inherent characteristics, biological properties and/or features of the skin which occur in response to the treatment, such as, for example, skin shrinkage, birefringence, temperature, water content, pH, elasticity, color, thickness, texture, mechanical damping properties, blood vessels, pigmented lesions, hairs, hair follicles, wrinkles, etc.

Which positional parameter measurements, skin characteristic measurements and/or skin response measurements are made can depend upon particular measurement results or treatment requirements. For example, if the handpiece is moving very rapidly across the skin and treatment power is proportional to relative handpiece speed, then bulk heating of the tissue can be a concern. In this case, the sensors can be instructed by the controller to measure skin parameters that are associated with blistering due to over treatment. If movement is slow, bulk heating and blistering can be less of a concern and more of the processing power of the controller can be used to make more accurate measurements of velocity with the sensors instead. Detailed examples of the invention are described below.

In some examples, a distinction can be made between micro-dosimetry and macro-dosimetry measurements. Micro-dosimetry measurements are substantially limited to one or more zones that are about to be treated or have been treated by a pulse, a set of sequential pulses, or a set of simultaneous pulses. For example, measurement of an approximately 1.2 mm diameter area that is co-centered with an approximately 1 mm diameter area that is about to be treated is micro-dosimetry because the measurement is substantially limited to the region that is about to be treated with a future pulse or a future set of essentially simultaneous pulses. In contrast, macro-dosimetry measurements are used to evaluate larger areas of skin to produce an average measurement of regions that include both areas that are about to be treated (or that have just been treated) and adjacent regions. In some examples, a sensor is used to produce micro-dosimetry or macro-dosimetry measurements in accordance with the feedback loops of this invention.

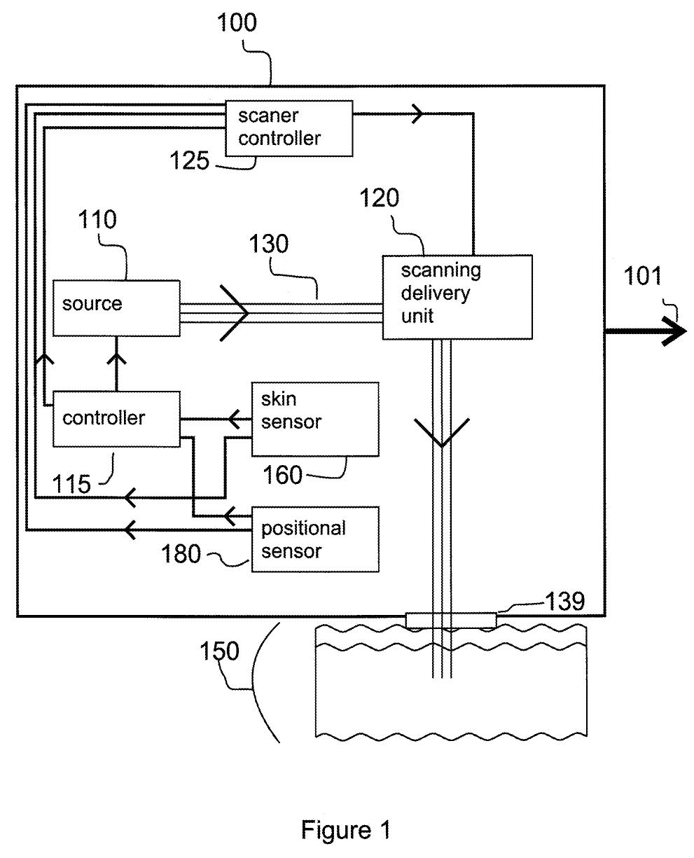

FIG. 1 is a diagram of an example of the invention showing a manually movable handpiece 100 that is configured to deliver electromagnetic treatment energy in a fractional manner to the skin 150 in the target region. The electromagnetic source 110 generates electromagnetic energy 130 that treats the skin. The controller 115 activates or adjusts one or more parameters of the electromagnetic source for the purpose of affecting treatment density. The handpiece 100 can contain a controller 115 that can comprise a computer, a radio frequency generator, and/or laser driver electronics. In other configurations, the controller 115 is located external to the handpiece 100 and is operably connected to the handpiece 100 to control treatment parameters so as to adjust treatment density. The system can also include an optional scanning delivery unit 120 that is operably coupled to a scanner control 125 that scans the electromagnetic energy 130 over the target region of the skin 150. An optional contact plate 139 that is mechanically coupled to the handpiece 100 can be used to make good electrical or optical contact with the skin 150 to enhance controlled delivery of the electromagnetic treatment energy 130, or to enhance the operation of a skin sensor 160 or a positional sensor 180. A positional sensor 180 measures positional parameters of the handpiece and a skin sensor 160 measures a skin response to treatment and/or a skin characteristic. Additional sensors for detecting positional parameters, skin responses and/or skin characteristics can be included in the system in order to measure different parameters, responses and/or characteristics, or in order to make multiple measurements of parameters, responses and/or characteristics in different parts of the target region of skin, or at different parts of the treatment process (e.g., one on the leading edge in front of the treatment beam and one on the trailing edge behind the treatment beam). These additional sensors are operably coupled to the controller so as to enable positional, response and/or characteristic feedback from the sensors to be used to control the treatment density.

While the operator manually moves the handpiece 100 in direction 101 or after the operator has manually moved the handpiece 100, the positional sensor 180 measures one or more positional parameters of the handpiece 100 and the skin sensor 160 measures a skin response to the treatment and/or a skin characteristic. The positional sensor 180 and the skin sensor 160 communicate with the controller 115 and/or with the scanner control 125.

In another example, while the operator manually moves the handpiece 100 in direction 101 or after the operator has manually moved the handpiece 100, one or more skin response sensors measure one or more skin responses to the treatment within the range of the sensor. The skin response sensor(s) communicate with the controller 115, the handpiece 100 and/or with the scanner control 125 in order for the controller 115, handpiece 100 and/or scanner control 125 to adjust the treatment density in real-time based on the skin response feedback. In yet another example, while the operator manually moves the handpiece 100 in direction 101 or after the operator has manually moved the handpiece 100, one or more skin characteristic sensors measure one or more skin characteristics of the skin within the range of the sensor. The skin characteristic sensor(s) communicate with the controller 115, handpiece 100 and/or with the scanner control 125 in order for the controller 115, handpiece 100 and/or scanner control 125 to adjust the treatment density in real-time based on the skin characteristic feedback.

The controller 115, the handpiece 100 and/or the scanner control 125 materially alter the treatment in real-time in response to the positional parameter measurements, skin characteristic measurements and/or skin response measurements provided by the sensors by adjusting the treatment density. Alternatively, the controller 115, handpiece 100 and/or the scanner control 125 can materially alter the treatment in real-time by adjusting (i.e., increasing or decreasing) the number of treatment zones created, the spacing of the treatment zones created, and/or the size of the treatment zones created. This adjustment of treatment zone number, spacing or size can be made in response to a positional parameter, a skin response, and/or a skin characteristic. An adjustment of treatment zone number, spacing or size can be made alone or in conjunction with an adjustment in treatment zone density.

In some examples, the feedback loops comprising the controller 115 and/or the scanner control 125 in combination with one or more sensors (e.g., the positional sensor 180, the skin sensor 160, etc.) can be used to provide automated control of treatment density, as well as to provide automated control of other treatment variables which may or may not affect treatment density, such as, for example, treatment location, treatment zone overlap, treatment energy, treatment depth, treatment power, treatment zone size, treatment zone pattern, treatment cooling (including pre-cooling and post-cooling), etc. The treatment density and/or the other treatment variables (e.g., treatment location, treatment zone overlap, etc.) can be controlled through adjustment of device parameters, such as optical focus, spot size, treatment pattern, pulse width, pulse energy, pulse timing, pulse frequency, laser power, laser wavelength, spray cooling volume, spray cooling timing, etc.

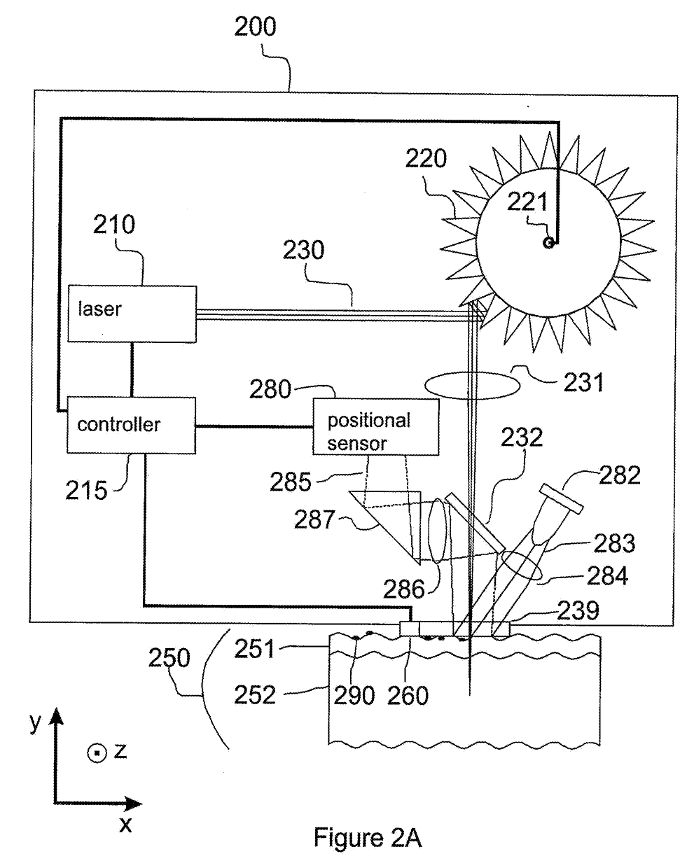

Optionally, the controller 115 can be operably connected to the scanner control 125, which can be helpful for reducing the number of wiring connections from the sensors. The controller 115 can serve the function of both the controller 115 and the scanner control 125 as shown in the example of FIG. 2A. For example, the functions of both the controller 115 and the scanner control 125 can be performed by a computer or a CPU operably coupled to a memory that stores a computer program. The sensors, including the positional sensor 180 and the skin sensor 160 can also be operably coupled or can be combined in a single component. For example, a CCD chip can be used to measure shifts in movement, skin characteristics and skin responses.

Detailed examples of several components in FIG. 1 are described in the examples given below. In one example, the electromagnetic source 110 delivers RF energy and the scanning delivery unit 120 comprises an electrical switching network comprising electrically controlled relays connected to multiple electrical contact pads in the contact plate 139 that is made of a nonconductive substance such as molded plastic. The scanning delivery system 120 can deliver a fractional treatment by delivering patterns of energy across the target region sequentially, or multiple relays can be activated to energize a plurality of treatment beams simultaneously so as to produce a plurality of treatment zones simultaneously.

In general, an electromagnetic source 110 is a radio frequency (RF) source, an optical source, or a combination of the two. A RF source generates electromagnetic energy with a frequency in the range of about 0.1 MHz to about 20 MHz, or in the range of about 0.5 MHz to about 8 MHz. An optical source generates light, which is defined for this application as electromagnetic energy with a wavelength in the range of about 300 nm to about 12,000 nm. In some examples, use of optical energy permits the energy to be directed accurately and easily to the desired target regions of the skin so as to deliver a fractional treatment. RF energy can also be used, particularly for applications where deeper penetration or targeting of particular buried layers of skin is desired. The choice of RF or optical energy can also be made to reduce interference with a chosen type of position sensor, skin characteristic sensor, and/or skin response sensor.

In one example, the electromagnetic source 110 is a laser and the electromagnetic energy 130 is a laser beam. Examples of lasers are Nd:YAG lasers, diode lasers, erbium fiber lasers, CO₂ lasers, Er:YAG lasers, Er:glass lasers, flashlamp-pumped lasers, free electron lasers, thulium fiber lasers, Raman shifted fiber lasers, dye lasers, gas lasers, Argon lasers, and ytterbium fiber lasers.

The skin characteristic and/or skin response can be measured by one or more skin sensors 160 employing one or more types of technology such as capacitive sensors, (hyper-) spectral imaging, terahertz imaging, optical coherence tomography, confocal microscopy, ultrasonic imaging, coherent detection, thermal detectors, thermal imaging, etc. In addition, one or more skin sensor(s) 160 can measure skin birefringence, skin water content, skin elasticity, skin mechanical damping parameters, skin color, skin features such as blood vessels and pigmented lesions, skin thickness, skin texture, wrinkles, etc. Other types of measurement technology and other dermatological features and tissue properties that can be measured will be apparent to those skilled in the art.

A mechanical mouse or roller wheel with an encoder can also be used as a positional sensor 180. Alternatively, a non-mechanical positional sensor which does not rely primarily on moving parts to measure positional parameters can be used. Non-mechanical positional sensors advantageously improve measurement reliability on slippery surfaces and reduce the chance of mechanical failure in comparison to mechanical positional sensors.

In one example of a non-mechanical positional sensor 180, coil sensors are used as described by Ben-Haim et al in U.S. Pat. No. 6,788,967, which is herein incorporated by reference. Three sensor coils that are mechanically coupled to the handpiece 100 in the appropriate orientations can be used to measure positional information, for example up to three dimensions and/or up to three angular orientations for the handpiece when the sensor coils are placed in the magnetic field generated by at least two radiators. Other geometries and numbers of radiators and sensor coils are possible for measurement of one-dimensional to six-dimensional positional parameters of the handpiece. Other non-mechanical positional sensors such as optical positional sensors are described below and can be detachable from the handpiece.

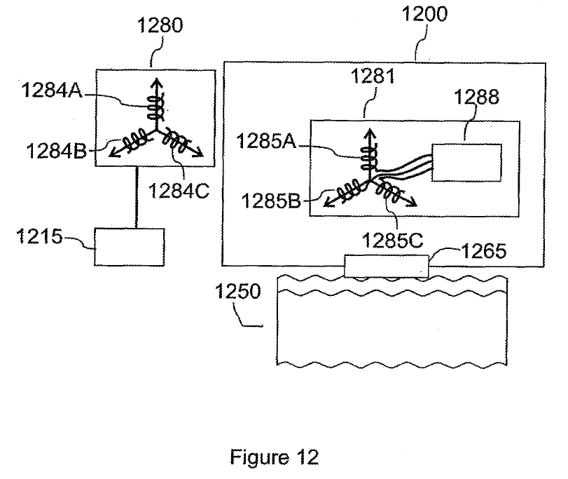

One example of the use of coil sensors is shown in more detail in FIG. 12. In FIG. 12, a magnetic positional sensor 1280 is located outside the handpiece 1200 and the magnetic source 1281 is attached to the handpiece 1200. The magnetic source 1281 can comprise three magnetic field source elements 1285A-C. The magnetic field source elements are arranged such that their axes span three-dimensional space. The axes can, for example, be directed in three mutually orthogonal directions. The magnetic positional sensor can comprise three magnetic sensor elements 1284A-C that can be located at a reference point and are arranged to span three-dimensional space.

In one example, each of the magnetic field source elements 1285A-C and each of the magnetic field sensor elements 1284A-C comprise a loop antenna that is tuned to a desired frequency, for example a frequency of about 10 kHz. The loop antennas 1285A-C for the magnetic field source elements 1282 can each be driven with a current source, for example an op-amp current source. Alternately, a single current source 1288 can be electronically switched to power each of the loop antennas of the magnetic field source elements 1285A-C sequentially. The system can be operated in the near field of each of the magnetic field source elements 1285A-C and each of the magnetic field sensor elements 1284A-C, but operation in the far field is also possible. The source elements 1284A-C can be sequentially powered in order to time division multiplex the source signals. The controller 1215 comprises receiver electronics for measuring the response detected by the magnetic field sensors. The receiver electronics portion of the controller can be collocated with the magnetic field sensor elements 1284A-C or can be integrated with the other electronics of the controller 1215. The controller comprises appropriate electronics to de-multiplex the received signals to identify the measured magnetic field intensity due to each of the source elements. To synchronize the systems, particularly in the case of time division multiplexing, a common clock can be used for the source and receiver electronics. Other configurations of source, receiver, multiplexing/de-multiplexing, and electronic systems will be apparent. For example, additional examples and refinements of appropriate magnetic field systems can be found in U.S. Pat. Nos. 4,613,866, 4,737,794, 4,742,356, and 5,307,072, each of which is incorporated herein by reference.

In another example, the magnetic field source elements 1285A-C are located at one or more reference points outside the handpiece and the magnetic field sensor elements 1284A-C are attached to the handpiece. The location of and direction of the treatment beam(s) emitted from the handpiece relative to the reference coordinate system is then measured. For treatment on the face, the handpiece 1200 can include the magnetic source 1281, and a small earbud that is placed within the ear of the patient can contain the magnetic positional sensor 1280. To improve accuracy and to determine whether the earbud has fallen out or shifted, a second magnetic positional sensor (not pictured) can be used, for example in the opposite ear of the patient. If there is a discrepancy between the redundant sensors, the system can alert the physician, using, for example, an audible alarm.

The choice of which of the magnetic source 1281 and the magnetic positional sensor 1280 is located at the reference point(s) and which is located at the handpiece 1200 can be chosen based on the sources of electromagnetic interference and objects of electromagnetic field distortion, such as metal plates. For the example above, it is anticipated that there is a scanning motor element, such as for example used in FIG. 2 to spin the scanner wheel 220 around axis 221 that generates a significant magnetic field. The effects of a scanning motor element on the measurement system can be reduced by locating the source in the handpiece instead of the sensor. In an alternate configuration, there can be no electromagnetic elements in the handpiece, for example, and the sensor can be optimally located in the handpiece and the source located at a reference point. In addition, the system can be calibrated empirically to compensate at least partially for any fixed elements that distort the magnetic field.

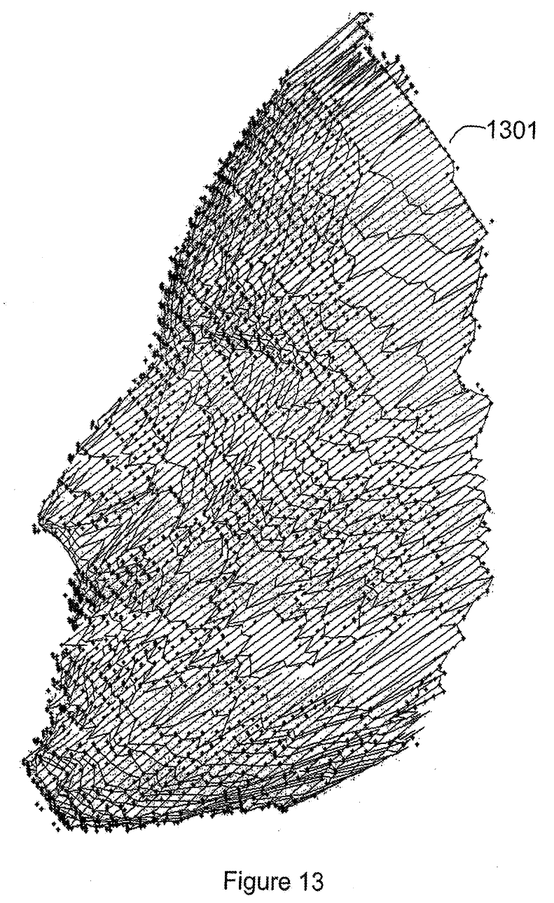

In one example of a magnetic field system as described in FIG. 12, a Polhemus Patriot™ digital tracker system (available from Polhemus, Colchester, Vt., USA) is used to measure the position of the handpiece relative to a reference point. An example of measurements created using this system are shown in FIG. 13, which shows a two dimensional (2D) projection 1301 of a three dimensional (3D) data set for half of a face.

In one example, one or more measured handpiece positional parameters include handpiece position or handpiece angle (angular orientation) or the time derivatives of these two parameters including handpiece velocity, handpiece acceleration, handpiece angular velocity, and handpiece angular acceleration. Handpiece positional parameters can be absolute or can be relative to the target region.

To enhance the serviceability of the apparatus and to allow handpieces to be interchanged and thus share expensive components, the handpiece can be detachable from one or more of the following: the electromagnetic source 110, the controller 115, and the scanner controller 125. To reduce the weight of the handpiece, these components can be located outside the handpiece. Alternatively, to enhance portability of the apparatus, these components can be included inside the handpiece.

The scanning delivery unit is configured to receive the electromagnetic energy 130 and deliver the electromagnetic energy 130 to the skin 150 regardless of where the other components are housed. For example, the electromagnetic source 110 can be a laser. The electromagnetic radiation can be coupled into an optical fiber, optical waveguide, or articulating arm for delivery to the handpiece. The handpiece can accept optical energy by using a fiber coupling or a fiber collimator. Similarly, it will be evident to those skilled in the art that the sensors 160 and 180 can be operably coupled to the controller 115, but do not need to be located inside the handpiece.

The controller 115 and scanner control 125 can be separate components as in FIG. 1 or can be combined as a single controller as shown in FIG. 2A.

In the example of FIG. 2A, a laser source 210 is used as the electromagnetic source. In this example, a manually movable handpiece 200 is configured to deliver an optical beam 230 of electromagnetic energy to the target region of the skin 250. The handpiece 200 contains a controller 215 comprising a computer and/or laser driver electronics. The controller 215 controls an optical source 210 and a scanning delivery unit 220 to affect one or more parameters such that treatment density is materially affected. The optical source 210 generates an optical beam 230 that is directed to an optional scanning delivery unit 220. The scanning delivery unit 220 deflects the laser beam 230 to different treatment zones on or within the skin 250 as will be described in greater detail below. For clarity, only one beam position is shown in FIG. 2A. A dichroic mirror 232 and a contact plate 239 that are substantially transparent at the wavelength of the laser beam 230 can advantageously be included in particular examples. The deflected laser beam 230 is delivered through the dichroic mirror 232 and contact plate 239 to the skin 250. A beam delivery lens 231 can be used to focus the deflected beam 230 within the epidermis 251, dermis 252, or other layers of the skin 250. The focal point of the optical beam 230 can be below the skin surface or the beam can be diverging or collimated as it enters the skin 250. The skin sensor 260 (i.e., skin characteristic sensor and/or skin response sensor) is mechanically coupled to the handpiece 200 and measures a skin characteristic and/or a skin response to treatment.

In the example of FIG. 2, the positional sensor 280 measures the position of the handpiece relative to the surface of the skin 250. In alternate examples, the positional sensor 280 can measure position, velocity, and/or acceleration of the handpiece relative to the surface of the skin 250. An illumination source 282 emits illumination 283 that is collimated by an illumination delivery lens 284 for delivery to the surface of the skin 250. Collimating the illumination 283 increases alignment tolerances, improves uniformity of the illumination on the skin surface, and allows the illumination source 282 to be placed further from the target region than would otherwise produce a uniform profile of illumination 283 at the surface of the skin 250. The illumination 283 is scattered from the surface of the skin 250 or from a contrast enhancing agent 290 that is placed into or onto the skin 250. The spectral reflectivity of the dichroic mirror 232 and the reflective prism 287 are designed to substantially reflect the wavelength of the scattered illumination 285. A detector lens 286 is placed in the optical path from the skin to the positional sensor 280 to image the surface of the skin 250 on the optical positional sensor 280. Examples of optical positional sensors 280 include an optical mouse chip (Agilent Technologies, Palo Alto, Calif., USA), a CCD camera, or an optical sensor array of at least two sensor elements. The optical sensor array can have at least 25 sensor elements, arranged as a 5×5 array in order to have sufficient resolution to accurately quantify a range of velocity resolutions easily. In one example, this optical positional sensor can be silicon-based so that it can be manufactured cheaply using bulk manufacturing processes and cheap material sources that have been developed for the electronics industry. Other configurations will be evident to those skilled in the art.

In FIG. 2A, the direction 201 of handpiece motion (not shown) is essentially perpendicular to the plane of the page. FIG. 2B illustrates a side view of the handpiece that shows the direction of motion 201 of the handpiece 200. For simplicity, internal elements of the handpiece 200 are not shown in FIG. 2B. The handpiece 200 is manually moved by the operator in direction 201 while the positional sensor 280 measures one or more positional parameters of the handpiece and the skin sensor 260 measures one or more skin characteristics and/or skin responses to treatment. The positional sensor 280 and the skin sensor 260 communicate with the controller 215. Alternatively or additionally, more than one skin sensor can communicate with the controller 215. In response to the measurements provided by the sensor(s), the controller 215 adjusts the optical treatment parameters so as to adjust the treatment density in real time to materially affect the photothermal treatment. For example, the rate of laser firing can be adjusted to be proportion to the velocity of the handpiece 200 to create a predefined treatment density. As a further example, the density of treatment zones produced in a portion of skin can be adjusted in response to the measurements, such as, for example, to reduce the density of treatment zones produced in the skin near a blood vessel so as to reduce side effects such as bleeding. As yet a further example, the density of treatment zones produced, as well as the treatment zone size, number, pattern, spacing, etc., can be adjusted in response to the measurements.

An example of a skin sensor 260 (i.e., a skin response sensor and/or a skin characteristic sensor) is a capacitive sensor as shown in FIGS. 2A, 2B, and 2C. The capacitive sensor 260 can measure the level of desiccation of selected layers of the skin due to treatment. The measurements from the capacitive sensor 260 can be used to calculate the proper treatment parameters for the treatment and make adjustments to the treatment density using the controller 215. The capacitive sensor 260 can also be used to evaluate whether a region of skin has blistered. By imaging the junction between the dermis and the epidermis, the capacitive sensor can determine whether separation of the dermis and epidermis has occurred. In other examples, sensors for measuring or imaging skin resistivity can be used as skin sensors 260 to evaluate blistering and skin moisture content. A capacitive sensor array that is commonly used for fingerprint measurements is an example of a sensor that can be used as a capacitive sensor 260.

FIG. 2C shows a treatment pattern comprising separated microscopic treatment zones 256 that can be created with this approach as the handpiece 200 is moved across the target region 257 in the direction 201. In this example, separated microscopic treatment zones 256A, 256B, and 256C can be created in the skin as described in the co-pending and co-owned U.S. application Ser. Nos. 10/367,582, 10/751,041, 10/888,356, and 11/158,907, which are herein incorporated by reference. In one example, the treatment zones 256 can be created in a predefined pattern that is invariant with the relative velocity or acceleration of the handpiece 100. Other patterns will be evident to those skilled in the art. Substantially uniform treatment density can be created by appropriately choosing optics, treatment parameters, and laser pulse timing. Alternatively, the capacitive sensor 260 (i.e., skin response sensor and/or skin characteristic sensor) can provide feedback to the controller 215 so that the treatment density can be adjusted in response to measurements provided by the sensor(s). For example, the capacitance sensor 260 can function as a skin response sensor to provide feedback to the controller 215 to increase or reduce the density of microscopic treatment zones 256 or to increase or reduce the treatment power in response to under-treatment or over-treatment.

In an alternative example, the treatment pattern can be intentionally varied according to a predefined algorithm where treatment density is varied in real time in response to changes in the velocity or acceleration of the handpiece and where the treatment pattern is not predefined. For example, the treatment density can be controlled in real time by the user by appropriately adjusting the position, velocity, or acceleration of the handpiece. In some treatments, it is desirable to allow the operator to have control over the treatment density through the use of velocity. For example, if the user treats quickly, the system can be configured to allow a higher treatment density, which in turn can be measured by the skin sensor 260. If the user treats slowly, then the treatment density can be reduced. Thus, the user is able to control the treatment density simply by changing positional parameters of the handpiece. Thus, the treatment density, as well as treatment pattern, treatment intensity, and other treatment parameters are not predefined, but can be defined through an automated response to measured positional parameters, to measured skin response, to measured skin characteristics, or to combinations thereof. An electronic or computer interface (not pictured) can be provided to allow switching on or off different modes of user control.

In another example, a treatment status map is displayed on a monitor (not shown) for the user or the patient to observe. The positional sensor 280 can be used to measure the location within the target region of the tissue response that is measured by the skin sensor 260. In this way, a map can display which parts of the target region have been treated and how each part of the target region has responded to treatment. The user can take the information on this map to make treatment uniform over the entire target region or to have treatment vary in a desirable manner such as treating areas with deep wrinkles more densely than less wrinkled areas. Alternatively, the system can be configured to automatically reduce treatment density or disable treatment in the regions that have already been adequately treated as the user continues to move the handpiece over the target region. A picture or schematic representation of the target region, such as a line drawing of a face for treatment of wrinkles on the face, can be used as a background for a computer display of the map of the treatment response measurements.

The use of a positional sensor 280 and/or a skin sensor 260 (i.e., a skin response sensor and/or a skin characteristic sensor) to create a map can be used beneficially, particularly with small beam sizes less than 1 mm in their smallest dimension. Using such a map, treatment can be turned on or off, or the density of treatment increased or decreased, based on whether treatment has covered that area or not, based on the presence or absence of skin features such as wrinkles, blood vessels, pigmented lesions, etc., or based on the presence or absence of an exogenous pigment. The advantage of using a beam size of less than 1 mm is that the granularity of the beam size for treatments that are visually apparent after treatment will be less noticeable for such small beam sizes. Thus, the use of a positional sensor 280 and/or a skin sensor 260 is particularly suited to fractional treatment and/or treatments with a small beam size of less than 1 mm.

Controller 215, optical source 210, and other components can be external to the handpiece 200 instead of being included inside the handpiece as illustrated in FIG. 2A. The optical beam 230 can propagate to the handpiece through free space, through an articulated arm, or through a waveguide, such as an optical fiber. The handpiece 200 can be mechanically separable from or mechanically separate from the external components and the handpiece 200 can be configured to receive the optical beam 230 and/or the signal from the controller 215.

In one example, the electromagnetic source 210 is a single mode pulsed erbium doped fiber laser with a peak output power in the range of about 5 W to about 50 W and a wavelength in the range of 1.52-1.62 μm. This laser source can be focused to an optical spot size in the range of about 30 μm to about 600 μm or about 60 μm to about 300 μm on the surface of the skin. Pulse energies in the range of about 2 mJ to about 100 mJ or in the range of about 8 mJ to about 20 mJ can be used for these ranges of optical spot size, wavelength, and power. This example does not include surface skin cooling, but such cooling can be included if desired to reduce damage to the epidermis and dermal-epidermal junction.

The scanning delivery unit 220 used in this example is a scanner wheel rotating at least 360° around an axis 221 as described in detail in U.S. application Ser. No. 11/158,907, which is incorporated by reference herein. Other scanner types will be apparent to those skilled in the art. For example, galvanometer scanners, pseudo stationary deflection (PSD) scanners as described in co-pending and co-owned U.S. application Ser. No. 10/750,790, which is also incorporated by reference herein, polygonal scanners, light valves, liquid crystal display (LCD) screens, micro-electromechanical system (MEMS) based reflective scanners, and translation stages can be used for the scanning delivery unit for delivery of optical energy. Multiple scanning delivery units can be used in such systems to control multiple axes of deflection. For example, two galvanometer scanners can be used in series to scan the laser beam in two directions to cover an area on the surface of the skin 250. Alternatively, single scanning units can cause beam deflection in two directions as described in detail in U.S. application Ser. No. 11/158,907.

One algorithm that can be used to control operational parameters of the scanning delivery unit 220 is to adjust the rotational speed of a double or single wheel PSD scanner and the laser firing rate in proportion to the velocity of the handpiece. This allows microscopic treatment zones of fractional resurfacing to be placed in a predefined density on the skin.

Another algorithm for controlling treatment is to adjust the firing of the laser in approximate proportion to the relative velocity of the handpiece to create a predefined treatment density. A uniform density of treatment zones distributed across a target region by overlapping or abutting treatment zones can also be achieved. For example, if the scanner 220 shown in FIG. 2A is controlled to spin at a constant angular velocity as the handpiece 200 is moving across the surface of the skin 250, the laser firing can be pulsed to create the desired treatment density by firing the laser only when it is aligned with a particular facet of the scanner that creates the desired treatment density. Not every facet needs to be used. For a particular velocity, every facet can be used. If the velocity is reduced by a factor of three from this velocity, then only every third facet can be used to keep the same density. In one example, the algorithm maintains a uniform density of treatment zones within the target region. Configurations which spin the scanning wheel 220 at a constant angular velocity can be used. Alternatively, configurations which require the angular velocity of the scanning wheel 220 to be proportional to the speed of the handpiece 200 can be used, although such configurations can increase the complexity of the motors, associated drive electronics, and encoders that are used to accurately control the angular velocity of the scanning wheel 220.

In another example, the scanner wheel 220 can be run at a velocity that drags the optical beam 230 across the target region. This wheel velocity can even be in the opposite direction of the direction that would compensate for movement of the handpiece. This intentional dragging of the optical beam 230 across the surface of the skin 250 can be created with either variable-velocity or fixed-velocity scanner systems. With the fixed-velocity system, for example, the pulse duration of the laser beam can be adjusted according to the velocity of the handpiece 200 such that the optical beam is dragged across the skin by approximately the same distance with each pulse. By changing the angular velocity of the scanner wheel 220 or by changing the pulse duration for the optical beam 230, the distance over which the optical treatment occurs for each pulse can be changed. The controlled dragging of the optical beam can, for example, be used to increase the fill factor for a fractional resurfacing treatment by making each microscopic treatment zone larger by increasing the distance over which optical treatment occurs. Similarly, the controlled dragging of the optical beam can, for example, be used to increase the treatment density for a fractional treatment by increasing the number of treatment zones created in the target region of skin. As the velocity of the handpiece 200 is reduced, the increased pulse duration prescribed by this algorithm can cause a reduction in treatment density as measured by the skin sensor 260. Therefore, it can be desirable to increase the pulse energy to keep the tissue response the same.

The contact plate 239 beneficially reduces optical scattering from the skin surface for the treatment beam by creating a smooth surface that can be used to precisely and reproducibly position the skin relative to the focus depth of the optical beam 230. The contact plate 239 can also act as a thermal heat spreader or can conduct heat away from the surface to actively cool the skin when connected to a cooling source (not shown). The contact plate 239 and dichroic mirror 232 can comprise sapphire, fused silica, borosilicate glass, transparent plastic, or other transparent materials. The contact plate 239, dichroic mirror 232, and other optical components can have optical coatings applied on one or more sides to increase the efficiency of energy delivery into the skin or to enhance the reflectivity or transmission of the illumination 283 from the illumination source 282.

In some examples, the contact plate 239 can be undesirable and can be omitted. For example, in ablative laser treatments, it can be desirable to have the surface of the skin be mechanically free to enhance the ablation response of treatment.

To enhance the ability of the optical positional sensor 280 to read the positional parameters of the handpiece 200, a contrast enhancing agent 290 can be applied onto or into the skin 250. For example, uniform application of a dye to the surface of the skin 250 can preferentially decorate certain features, such as skin wrinkles or hair follicles, to create shapes that can be detected as objects by the positional sensor 280. The contrast enhancing agent 290 must be non-toxic when applied onto or into a patient's skin in amounts suitable for adequately enhancing measurements by the positional sensor 280. In one example, the contrast enhancing agent and the materials and geometry chosen for the handpiece 200 and contact window 239 allow the handpiece 200 to slide easily over the surface of the skin 250.

Examples of contrast enhancing agents 290 are carbon particles, India ink, and FD&C Blue #1. Many other dyes, inks, particulates, etc., can be used as contrast enhancing agents when applied to the skin and when used with the appropriate positional sensor 280. The wavelength illumination source 282 can be chosen to maximize the signal to noise ratio of the measurement of the positional parameters of the handpiece 200. For example, a red LED with a peak wavelength in the range of about 600 nm to about 640 nm can be used with FD&C Blue #1.

In many cases, the contrast enhancing agent can be chosen such that it has a low absorption of the treatment energy or of the treatment wavelength in the case of optical treatment energy. In this way, the contrast enhancing agent will not interfere with the deposition of the treatment energy in the target region. In some cases, the contrast enhancing agent is chosen such that a measurable or observable parameter changes in response to the treatment energy. A change in the contrast enhancing agent can be used to determine where treatment has occurred, which allows the treatment to be touched up in areas where it is not even or uniform.

It is desirable to choose a contrast enhancing agent 290 that can be removed without abrasive or harsh scrubbing. Alternatively, a removal facilitation substance (not shown) can be applied prior to application of the contrast enhancing agent 290 to allow the dye to be removed more easily. Dimethicone, urea, and arginine are examples of removal facilitation substances. These substances can be applied prior to the contrast enhancing agent 290 to facilitate subsequent removal of the contrast enhancing agent 290. These substances can be applied using common solvents such as water, alcohol, or oil. Concentrations of the removal facilitation substance can be used, for example, in the range of about 0.001M to about 0.1M.

It is desirable to choose a contrast enhancing agent 290 that is not clearly visible when illuminated with typical room light and/or sunlight. Contrast enhancing agents 290 are said to be “hypo-visible” if and only if the contrast enhancing agent is not readily visible on otherwise bare skin with the naked eye when illuminated with about 400 nm to about 650 nm light when the contrast enhancing agent 290 is applied such that the response of the detector 280 is beneficially and substantially enhanced when using an illumination wavelength from about 300 nm to about 400 nm or from about 700 nm to about 1100 nm. The use of hypo-visible contrast enhancing agents 290 is desirable because the contrast enhancing agent 290 will be less visible after treatment even if not all of the contrast enhancing agent 290 is removed from the target region.

Many fluorescent inks, lakes, dyes, and particulates are examples of hypo-visible contrast enhancing agents 290. Fluorescing agents are desirable because the wavelength of illumination can be filtered by the dichroic mirror 232 or by other optical components or coatings while the throughput of the fluorescent emission wavelength is maximized to improve the signal to noise ratio of the positional sensor 290. Polymer (PMMA) encapsulated fluorescent dyes are commercially manufactured by NewWest Technologies (Santa Rosa, Calif., USA). Other fluorescent materials include collagen, elastin, FD&C Orange No. 5, flavin adenine dinucleotide, flavin adenine mononucleotide, folic acid, niacin, nicotinamide, reduced nicotinamide adenine dinucleotide (NADH), porphyrins, pyranine (FD&C Green No. 7), pyridocine hydrochloride, quinine sulfate, riboflavin, riboflavin phosphate, tryptophan, uranine (fluorescein), or combinations thereof. The absorption and emission spectra for these substances are well published in the art. Other fluorescent materials that are well known in the art can also be used as the contrast enhancing agent 290, for example Carbazine, Coumarin, Stilbene 3, Kiton Red, etc.

As the intensity of fluorescent emission of pyranine varies with pH, pyranine can be used to evaluate changes in barrier function and alert the user or automatically stop treatment or reduce treatment intensity if a break in the stratum corneum or a rupture of the skin occurs during treatment. Thus, the contrast enhancing agent 290 can also be used to improve the signal to noise ratio of the skin sensor 260.

Indocyanine green (ICG) is an example of a contrast enhancing agent 290. Most contrast enhancing agents 165 can be diluted with water or other solvents to make them easier to apply or cheaper to use. The peak wavelength of ICG varies depending on the solvent and the concentration of ICG. For example, in water, ICG has an IR absorption peak at approximately 700 nm for high concentrations (e.g. about 129 μM to about 1290 μM) and at approximately 780 nm for low concentrations (e.g. about 6.5 μM to about 65 μM). For ICG in blood plasma, there is an absorption peak in the range of approximately 790 nm to approximately 810 nm across a broad range of concentrations (about 6.5 μM to about 1290 μM). In general, ICG typically has an absorption peak in the range of about 650 nm to about 850 nm for most solvents. ICG also has absorption peaks in the UV range. ICG does not have a strong absorption peak in the range of about 400 nm to about 650 nm, which makes it difficult to see with the naked eye. Thus, ICG is an example of a contrast enhancing agent that has low visibility to the human eye, but is easily discernable to a silicon based optical detector when illuminated appropriately. In non-fluorescing contrast enhancing agents, the wavelength (or wavelength range) of illumination can be chosen to be in a region where the peak absorption of the contrasting agent is at least about 3 times, or at least about 10 times, stronger or weaker than that of skin. It is also desirable to have the peak absorption of the contrasting agent in the chosen wavelength (or wavelength range) to be at least about 3 times, or at least about 10 times, stronger than the peak absorption within the wavelength range of about 400 nm to about 650 nm.

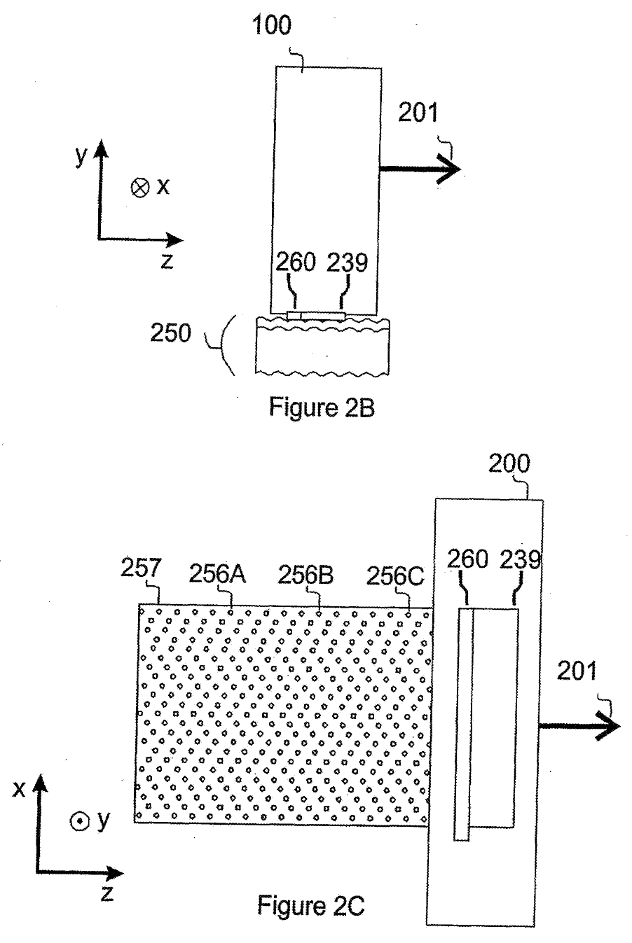

The contrast enhancing agent can also be applied in a pattern. The pattern can comprise a uniform grid of identical FIGS. 391 in the target region 357 as illustrated in FIG. 3A. The pattern can comprise a non-uniform pattern of identical FIGS. 392 in the target region 357 as illustrated in FIG. 3B. The pattern can comprise a non-uniform pattern of a plurality of different FIG. 393 in the target region 357 as illustrated in FIG. 3C. Contrast enhancing agents can be applied using stamps, rollers, sprays, stencils, or with agent-soaked gauze pads.

Patterns of contrast enhancing agents can also be attached to the skin using adhesives as used in temporary tattoos. As in a temporary tattoo, a pattern can be created by printing a contrast enhancing agent on or embedding a contrast enhancing agent in an adhesive that attaches to the skin. The adhesive has the advantage of being easier to remove than many of the contrast enhancing agents that can be included in or on the adhesive. Lakes of Food and Drug Administration (FDA) approved colors such as FD&C Blue #1 (also packaged as Optiguide Blue by Reliant Technologies, Mountain View, Calif., USA) can be embedded in a polymer-based tattoo adhesive and painted onto the skin. Following treatment, these adhesive based patterns can be removed with alcohol and light scrubbing. The use of adhesive also allows the use of contrast enhancing agents in doses that would otherwise be toxic to the skin because the adhesive can be designed to provide a barrier between the skin and the contrast enhancing agent.

Alternatively, contrast enhancing agents can be suspended in sugar-based or gel based solutions without patterning. These solutions can desirably be made viscous so that they do not drip outside the target region.

Instead of applying a pattern of figures with a contrast enhancing agent, the laser treatment zones can form a pattern of figures that is used to enhance the response of the positional sensor 280. For example, a CO₂ laser can ablate portions of the skin to create a pattern of ablated areas interspersed inside non-ablated areas. This pattern can be illuminated with a light emitting diode (LED) to provide visible features that enhance the signal to noise ratio of an optical mouse chip functioning as a positional sensor 280.

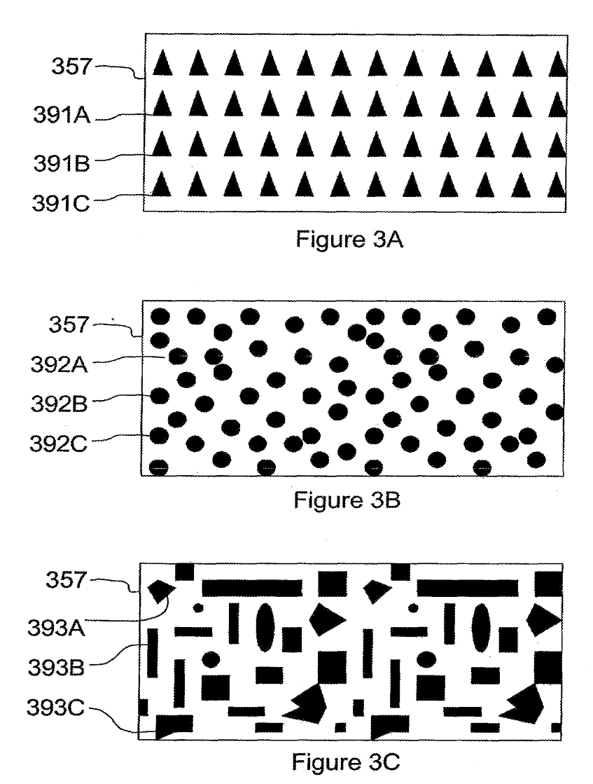

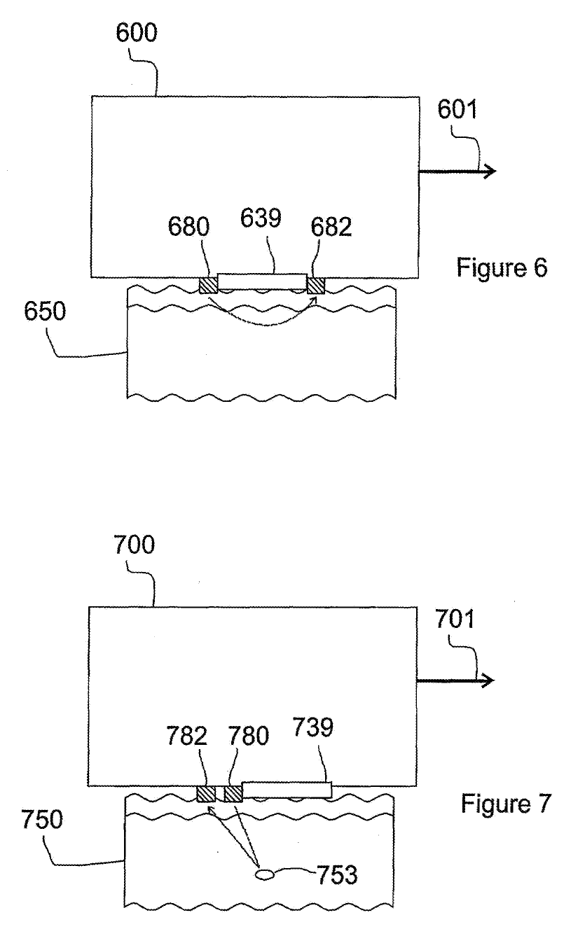

Other examples of positional sensor 280 are illustrated in FIGS. 4-7. Other examples of skin sensor(s) 260, (i.e., skin response sensor(s) and/or skin characteristic sensor(s)) are illustrated in FIGS. 8-11. Using one or more of these sensors, (i.e., positional sensors, skin response sensors, and/or skin characteristic sensors), different measurements can be made to optimize tissue treatment density. Treatment density can be kept constant or maintained within defined ranges by the controller 215 which appropriately adjusts treatment parameters of the electromagnetic source 210 and the scanning delivery unit 220. Alternatively, treatment density can be changed by the controller 215 based on feedback from the sensor(s).

The positional sensors and skin sensors shown in FIGS. 4-11 can be added to or substituted into the example shown in FIGS. 1 and 2. As will be apparent to one skilled in the art, many of these systems can be easily designed such that the region sensed by the skin sensor is coincident with the region measured by the positional sensor and the target region being treated. In situations where it is not desirable to have multiple sensors coincident or where these multiple types of sensors interfere, the skin sensor(s), can be displaced along the x, y, or z directions relative to each other.

While the examples illustrated in FIG. 2 shows delivery of optical energy to the target region, monopolar or bipolar radio frequency (RF) energy can also be used in place of optical energy by replacing the contact plate 239 with a contact plate, contact electrodes, or needle electrodes that are configured to deliver RF energy to a desired target region under the control of a controller 215 that comprises a RF generator.

FIG. 4 illustrates another example of the invention. In this example, the positional sensor is implemented as one or more sets of accelerometers 480 and 481 that are mechanically coupled to the handpiece 400. The sets of accelerometers 480 and 481 can be attached to the inside or outside of the handpiece 400. A set of three accelerometers 480A, 480B, and 480C can be used to measure changes in velocity in each of the three coordinate planes. The one or more sets of accelerometers 480 and 481 can communicate with a controller 415 that controls the operational parameters of an electromagnetic source 410. The electromagnetic source 410 emits electromagnetic energy 430, which is delivered to the skin 450 through a contact plate 439. The configuration illustrated in FIG. 4 can also include a scanning delivery unit (not shown), as illustrated in FIGS. 1 and 2.

As shown in FIG. 4, a pair of accelerometers can be used to measure angular acceleration in each of the three rotational directions. For example, accelerometers 480A and 48 1A measure the angular acceleration around a rotational axis parallel to the z axis, accelerometers 480B and 481B measure the angular acceleration around a rotational axis parallel to the x axis, and accelerometers 480C and 481 C measure the angular acceleration around a rotational axis parallel to the y axis. Accelerometers 480B and 481B are displaced from each other along the z axis direction and are drawn as overlapping in FIG. 4. Alternatively, gyroscopes can be used to measure angular acceleration of the handpiece. MEMS based accelerometers and gyroscopes are sold by several suppliers (e.g. Kionix, Inc., Ithaca, N.Y.).

Measurements of acceleration or angular acceleration can be integrated in time to produce measurements of velocity and position or angular velocity and angular position. In many configurations, an initial calibration and periodic recalibrations can be required to reset the reference velocity, angular velocity, position, and/or angular position.

Accelerometers measure absolute positional parameters of the handpiece 400 rather than relative positional parameters of the handpiece 400 with respect to the target region of the skin 450. If relative positional parameters are desired, accelerometers can be used when the target region is immobilized or when absolute movement of the target region is insignificant. Alternatively, the absolute movement of the target region of the skin 450 and the absolute movement of the handpiece 400 can both be measured and the relative motion between the handpiece 400 and the target region of the skin 450 can be calculated.

Relative measurements of angular position can be used to provide feedback to the system and disable the laser unless the relative angle of the handpiece is within a certain angular range relative to the surface normal from the surface of the target region. This can be useful, for example, to align properly a cooling spray and a treatment laser beam on a target region. Absolute measurements of angular position are useful if the handpiece 400 has components that are sensitive to gravity, such as fluid-filled cavities that leak if turned upside down. Relative measurements of position can be used to measure distance between locations for pulsing the electromagnetic source 410.

Absolute or relative measurements of velocity, acceleration, angular velocity, and angular acceleration are useful for evaluating whether the handpiece has been dropped or has suddenly slipped in an uncontrolled way, which might lead to undesired treatment outside the desired target region. A combination of relative positional parameter measurements and absolute positional parameter measurements can be used to measure movement of the patient. For example, if the patient suddenly moves, the difference between the relative acceleration and the absolute acceleration measurements can be significant. In any of the situations described in this paragraph, the controller 415 can temporarily disable the electromagnetic source 410 to prevent treatment in areas that are not desired by the user.

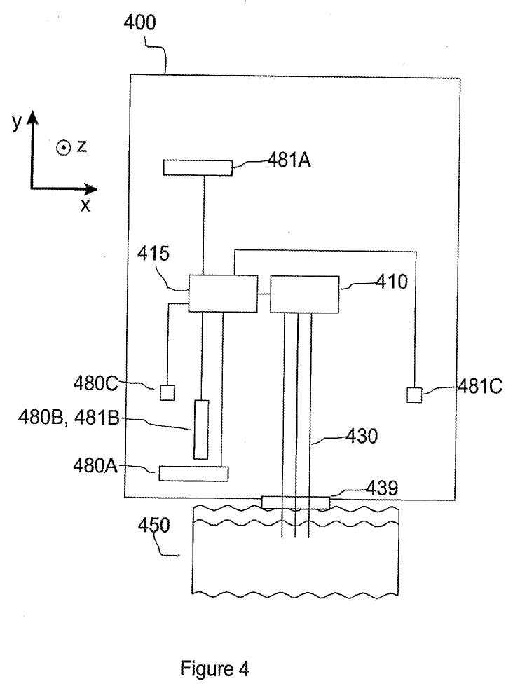

FIG. 5 illustrates another example of the invention. In this example, the positional sensor comprises at least two pairings of transmitter and receiver that conduct either unidirectional or bidirectional wireless communication. The transmitters 580A-C are positioned to transmit signals to one or more receivers 581A-B that are mechanically coupled to the handpiece 500. The signals from the receivers are received by the controller 515, which uses time of flight measurements or phase measurements to calculate the distance between each pairing of transmitter and receiver. These distances can be used to calculate selected positional parameters of the handpiece, which can be done by the controller 515. The controller 515 can be operably connected to other components of the handpiece such as the electromagnetic source 110, the scanner control 125, or the scanning delivery unit 120 as shown in FIG. 1. These can be located inside or outside the handpiece 500 and, for simplicity, are not shown.

The number and location of transmitters and receivers determines the positional parameters that can be measured. For measuring the position of the handpiece in three dimensions, three transmitters and one receiver can be used. For measuring the position of the handpiece in up to three dimensions and also measuring the angular position for up to three independent angular directions, a second receiver can be used. For measuring all three dimensions and all three handpiece angles, three transmitters and three receivers can be used in order to have redundancy. A simple apparatus comprises two transmitters and one receiver. This apparatus can be used to measure the positional parameters of a handpiece in two dimensions along a predefined surface. In an alternate configuration, two receivers are used with one transmitter to produce the same measurement. The particular geometry and locations of transmitters and receivers can be generalized by one skilled in the art.

For simplicity in the examples described below, receivers are located on the handpiece and transmitters are located inside the target region 557 or are mechanically coupled to the target region 557 such that the measured positional parameters of the handpiece will be relative to the target region and not absolute measurements. Other configurations can be used if absolute measurements are desired. Light based or other electromagnetic communications systems can be used for these types of systems as well.

In one example, three radio frequency transmitters are attached to a cap, such as, for example, made of cloth or latex for ease of use and low cost. For example, transmitters can be attached to electroencephalograph (EEG) caps for this purpose. This type of cap is useful for locating the handpiece when treating wrinkles on the forehead or periorbital areas of the face, for example, because the transmitters can be mechanically coupled to the target region. This type of cap can also be used with the coil measurement system described in the text for FIG. 1. In some examples, single chip receivers, similar to those commonly used in cell phones or global positioning satellite (GPS) tracking systems, are attached to the cap. Alternatively, sensors or receivers can be attached directly to the target region or to other areas of the body, such as the teeth, ears, nose, chin, etc., using adhesives. If the sensors are placed accurately in the same place for each treatment, for example on the same tooth, then overlay maps can be created to illustrate the regions that were treated with each treatment in a series.

One advantage of the accelerometer, magnetic, gyroscope, and transmitter-receiver based measurement systems is that they can easily be used in noncontact treatment mode (i.e., where the handpiece does not come in direct contact with the skin), which reduces the chance of skin movement during treatment and allows the handpiece to be held at different distances from the skin in order to manually adjust the beam size that is incident on the skin surface.

Multiple positional sensors can also be used, for example, to allow lower quality signals from each of the positional sensors. For example, an optical mouse type sensor can be used with a magnetic radiator coil measurement system. The combination of multiple sensors can also be used to shut the system down if large discrepancies were noted between or among the sensors. If different types of sensors were used, discrepancies can be used to provide additional information, for example, about whether the skin is being stretched. This information can be used to detect situations when the handpiece is not sliding properly and can be used to provide feedback to the system and reduce localized over- and under-treatments.

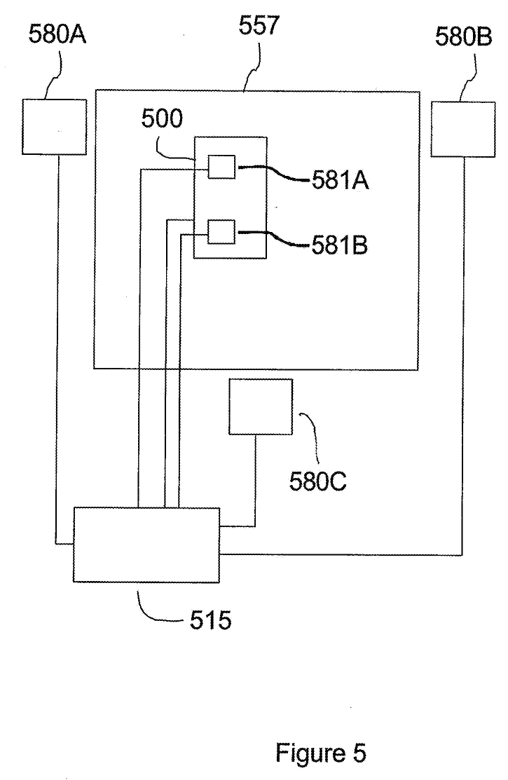

FIG. 6 shows another example of the invention wherein a manually movable handpiece 600 is configured to deliver optical energy to the skin. An ultrasonic transmitter 680 is positioned on one side of the contact plate 639 and an ultrasonic receiver 682 is positioned on the opposite side of the contact window. Time-of-flight measurements or phase measurements are recorded to measure the distance of propagation between the transmitter 680 and receiver 682. This can be used to measure velocity of the handpiece 600 in the direction 601 relative to the skin 650.

FIG. 7 shows an example of the positional sensor and a handpiece 700. A phased array of ultrasonic transmitters 780 is positioned on one side of the contact plate 739 and an ultrasonic receiver 782 is positioned on the same side of the contact window. The phased array 780 emits a directional ultrasonic beam that can be scattered or reflected from the surface of the skin or from one or more features 753 within the skin to the ultrasonic receiver 782. Using phase shift, time of flight, or Doppler frequency shift measurements, a controller (not shown) can be used to measure positional parameters of the handpiece 700 as it moves in the direction 701.

The ultrasonic transmitter-receiver pairs shown in FIGS. 6 and 7 can also be used as examples of the skin sensor 160 from FIG. 1, or of embodiments of a skin response sensor and/or a skin characteristic sensor, with the proper choice of frequency. Such a sensor can be used in conjunction with a velocity sensor to remove the changes in the measurement due to velocity.

FIG. 8 shows an example of the skin sensor 160 from FIG. 1, or of a skin response sensor and/or skin characteristic sensor. In this example, a polarized illumination source 862 is used to illuminate the skin 850 through an illumination lens 864 and through an optional transparent contact plate 839. A polarized imaging system comprising an imaging sensor 860, a polarizer 867, and an imaging lens 866 are used to image the birefringence of the target region of the skin 850. The imaging sensor 860 can then be operably coupled to the controller 115 shown in FIG. 1.

During certain types of photothermal treatment, dermal collagen is coagulated, which causes a loss of optical birefringence for the collagen. This change in birefringence can be measured by the imaging sensor 850 and can be used, for example, as the endpoint of a treatment pulse to control the duration of a treatment pulse. Additionally, this change in birefringence can be measured by the imaging sensor 850 and can be used to evaluate a skin response or characteristic which can then be used by the controller to control treatment density as well as other treatment parameters.

The polarizer 867 can be adjustable (automatically or manually) to make alignment easier or more precise or to allow comparison of cross polarization and parallel polarization images.

The example shown in FIG. 8 can also be used to measure skin shrinkage, such as, for example, by measuring the separation distance between two features on the skin before and after treatment. One or more imaging sensors 860 can be used. Shrinkage can also be measured using a single measurement by measuring the separation distance between individual treatment zones that start at a known distance. For example, an ablative CO₂ laser can place two marks at a set distance of about 15 mm and then the separation between these marks can be measured to determine skin shrinkage. Use of the polarizer 867 can be unnecessary for these measurements and the illumination source 862 can be unpolarized.

In another implementation of the skin sensor illustrated in FIG. 8, illumination can be used to increase the signal level of an optical skin sensor (i.e., skin response sensor and/or skin characteristic sensor). White light illumination can be used. Alternatively, sequential illumination with different color illumination sources can be used to capture images that are digitally processed to spectrally determine the response and/or characteristics of the skin. For example, illumination from a red LED at about 660 nm and green LED at about 555 nm can be used to capture, for which the absorption of melanin and blood are different. This will help to distinguish between treatment lightening response of pigmented lesions and of blood vessels. This can also be used to determine the presence of skin features such as blood vessels and pigmented lesions. Use of the polarizer 867 can be unnecessary for these measurements and the illumination source 862 can be unpolarized.

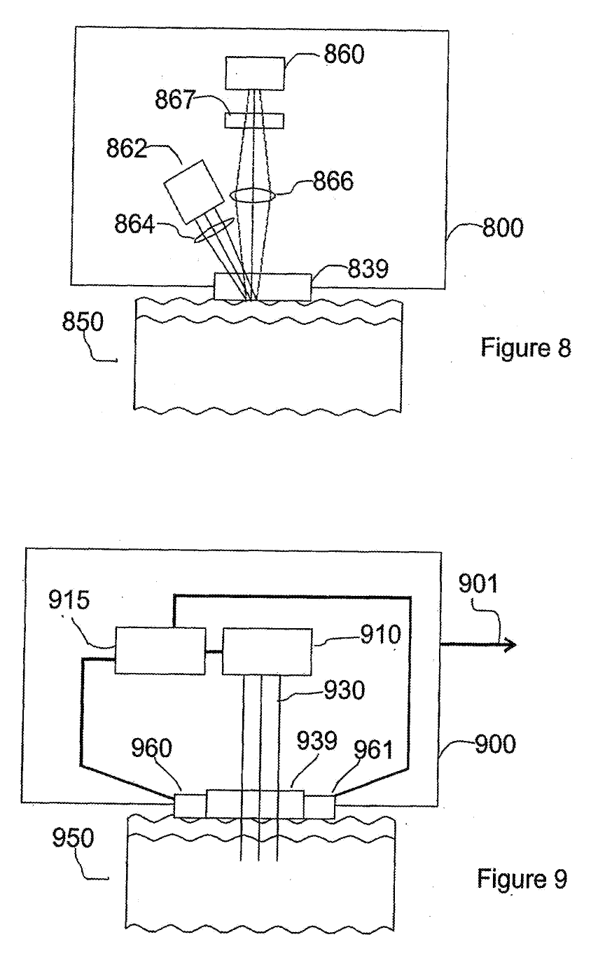

FIG. 9 shows an example of the invention that uses a plurality of skin sensors (i.e., skin response sensors and/or skin characteristic sensors) 960 and 961 to provide more information than is available from a single sensor. For example, one skin sensor 961 can measure the skin prior to treatment and a second skin sensor 960 can measure the skin after treatment. In this example, the two skin sensors 960 and 961 are operably coupled to a controller 915 that controls the treatment parameters of the electromagnetic source 910, including the treatment density. The electromagnetic source 910 generates electromagnetic energy 930 that is delivered in a fractional manner to the target regions of the skin 950 through a contact plate 939 as the handpiece is moved in a direction 901.

Using a skin response sensor 961 before treatment and another skin response sensor 960 after treatment allows the controller 915 to calculate the treatment density applied, or how the skin response has changed after treatment for a particular treatment density setting. The controller 915 can then make adjustments as appropriate to adjust the treatment parameters of the electromagnetic source 910 in order to adjust the treatment density. This feedback loop allows real time adjustment of treatment density.

An example of a feedback loop uses a first capacitive sensor 961 and a second capacitive sensor 960. Each capacitive sensor measures the percentage of skin that has been treated with a nonablative fractional resurfacing treatment. The first and second capacitive sensors 961, 960 are positioned in front of and behind the treatment window such that the first capacitive sensor 961 measures the percentage of skin that had been treated prior to the current pass of the handpiece and the second capacitive sensor 960 measures the percentage of skin that has been treated after the current pass of the handpiece over the target region. The difference between the measurements for the two sensors 960, 961 describes the percentage of skin treated during the current pass of the handpiece over the treatment region. The calculation of the percentage of skin treated during the current pass can be used, for example, to avoid over-treatment caused by bulk heating of tissue by reducing the laser treatment density when unusually high percentages are calculated. Other examples of appropriate feedback sensors 960, 961 are described in U.S. application Ser. No. 10/868,134, which is incorporated by reference herein.

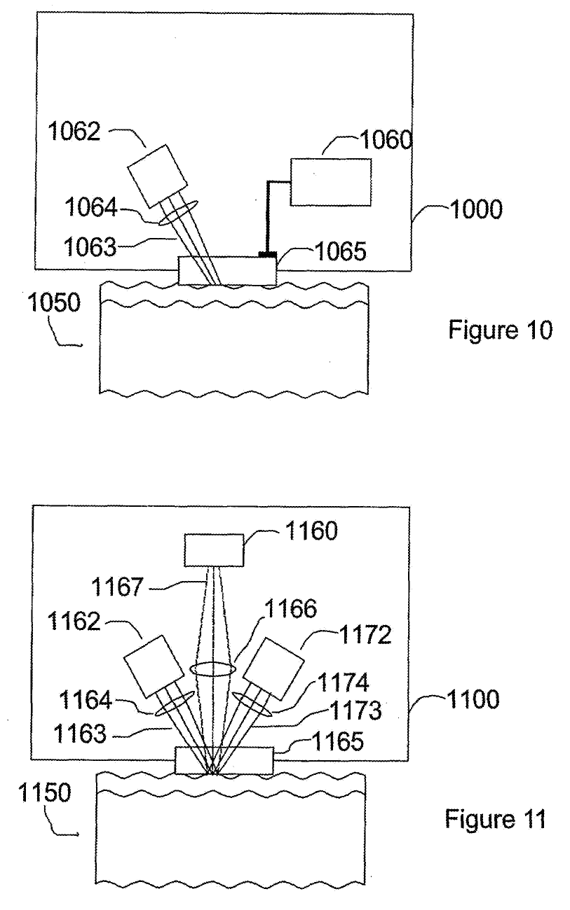

FIGS. 10 and 11 show other examples of a skin sensor 1060/1160 that is operably connected to a controller (not shown), which changes the treatment density in response to the measurements from the sensor. In one example, the sensor 1060/1160 is located inside the handpiece 1000/1100. In another example, the sensor 1060/1160 is not located inside the handpiece 1000/1100. In FIG. 10, a probe radiation source 1062 generates a probe beam 1063, such as, for example, with a pulse width of between about 0.5 ns and about 1000 ns or between about 5 ns and about 100 ns, that is absorbed by the skin 1050 to create a stress wave that propagates through the interface between a piezo-electric material 1065 and the skin 1050. The probe beam 1063 can pass through an optional probe beam delivery lens 1064 to focus the probe beam 1063 onto or into the skin 1050. The stress wave causes the piezo-electric material 1065 to generate an electrical signal that is measured by the electrical signal detector 1060 that is electrically connected to the piezo-electric material 1065.

The characteristics of the generated stress wave vary based on mechanical and optical characteristics of the skin. The probe wavelength can be chosen such that there is a difference in absorption within the skin between untreated and treated skin. Alternatively, the pulse conditions are chosen such that the mechanical response is different for treated and untreated skin. Thus, the stress wave that is created can be measured to determine whether the probed skin is approaching, has reached, or has exceeded a desired density of treatment. Examples of mechanical characteristics of the skin that can be probed using a stress wave include elasticity, tension, and mechanical damping of the skin.

The signature of the stress wave that is generated can be measured using several different techniques. One technique is illustrated in FIG. 10 and is described above. In this technique, a transparent contact plate 1065 made of a piezo-electric material, such as lithium niobate, generates an electrical signal in response to a mechanical stress wave. This electrical signal can be measured by an electronic signal detector 1060. Appropriate electronic signal detectors 1060 are well described in the art. The probe radiation source 1062 can be a Q-switched or mode-locked lasers. The laser can be a diode laser, a solid state laser, an Nd:YAG laser, a gas laser, etc.

A second technique for measuring the stress wave is to observe the change in reflectance pattern from a beam incident on the surface of the skin as shown in FIG. 11. In this configuration, a probe radiation source 1162 generates a probe beam 1163 that is absorbed by the skin 1150 to create a stress wave that propagates along the surface of the skin 1150. The probe beam can, for example, have a pulse width of between about 0.5 ns and about 1000 ns or between about 5 ns and about 100 ns. The probe beam 1163 can pass through an optional probe beam delivery lens 1164 and an optional contact plate 1165 if desired for optical or mechanical purposes such as focusing the probe beam 1163 or mechanically enhancing the propagation of the stress wave. A coherent illumination source 1172 generates a coherent illumination beam 1173 that can be focused or collimated onto the surface of the skin using an optional coherent illumination lens 1174. The coherent illumination beam 1173 is diffracted from the surface of the skin by the stress wave created on the surface of the skin 1150 to create a diffracted beam 1167. The diffracted beam 1167 can be imaged using an imaging lens 1166 onto an imaging detector 1160, such as a CCD camera.

The components 1162, 1163, and 1164 are similar to their analogs in FIG. 10 1062, 1063, and 1064 and can be made from the same components as described above.

The optional contact window 1165 can be comprised of a transparent material, such as fused silica or sapphire, through which the probe beam 1163 passes.

The probe beam 1163 is absorbed by the skin 1150 to create a stress wave in the skin 1150. As described above for FIG. 10, the features of the stress wave depend on the optical and mechanical parameters of the skin. Certain features, such as the period and damping of the stress wave, can be evaluated by measuring the diffraction pattern from the diffracted beam 1167 that is imaged on the surface of the imaging detector 1160.

The coherent illumination source 1172 can be a coherent source, for example a HeNe laser. The angle of the coherent illumination beam 1173 relative to the surface of the skin 1150 and the angle of the imaging system relative to the surface of the skin 1150 and relative to the coherent illumination beam 1173 can be aligned to maximize the measurement signal. Once a signal has been measured, the decay constant and resonant frequency of the stress wave can be measured with of the apparatii described by FIGS. 10 and 11. DC filtering can also be used to improve the signal to noise ratio of the detected signal.

With the techniques described in FIGS. 10 and 11, in one example, only the first reflected wave is measured and subsequent signals from scattering are temporally filtered. This reduces confusion from multiply reflected waves. This is similar to optical coherence tomography systems in which only the first reflected signal is used. Depending on the particular geometry of the apparatus, this apparatus can be used to measure bulk or localized optical and mechanical properties or characteristics of the skin, which are changed by the treatment. Alternatively, this apparatus can be used to measure bulk or localized optical and mechanical properties or characteristics of the skin, which are not changed by the treatment.

The examples presented here have all illustrated the use of these techniques on human skin. This invention is also applicable to treatment of other tissues of the body. For example, puncturing the surface of toenails for treatment of nail fungus, altering the soft palate for treatment of disorders such as sleep apnea and snoring, removing hair, delivering pharmaceuticals or nutriceuticals through the skin or mucosa, or treating heart tissue can all benefit from the use of this invention.

Although the detailed description contains many specifics, these should not be construed as limiting the scope of the invention but merely as illustrating different examples and aspects of the invention. It should be appreciated that the scope of the invention includes other embodiments not discussed in detail above. For example, in many of the examples above, lasers are used as the embodiment, but these can be generalized to RF, flashlamp, or other electromagnetic energy based treatments as well. Various other modifications, changes and variations which will be apparent to those skilled in the art may be made in the arrangement, operation and details of the method and apparatus of the present invention disclosed herein without departing from the spirit and scope of the invention as defined in the appended claims. Therefore, the scope of the invention should be determined by the appended claims and their legal equivalents. Furthermore, no element, component or method step is intended to be dedicated to the public regardless of whether the element, component or method step is explicitly recited in the claims.

All publications, patents and patent applications cited herein, whether supra or infra, are hereby incorporated by reference in their entirety.

In the specification and in the claims, reference to an element in the singular is not intended to mean “one and only one” unless explicitly stated, but rather is meant to mean “one or more.” In addition, it is not necessary for a device or method to address every problem that is solvable by different embodiments of the invention in order to be encompassed by the claims.

What is claimed is:

1. A method for controlled fractional skin treatment comprising:
manually moving a handpiece across a target region of skin;
directing electromagnetic energy via the handpiece toward the target region in a manner so as to provide a fractional treatment;
sensing at least one positional parameter, skin response and/or skin characteristic in the target region; and
automatically adjusting treatment density of the fractional treatment in real-time in response to the sensed at least one positional parameter, skin characteristic and/or skin response.

2. The method of claim 1, wherein the fractional treatment is delivered while the handpiece is in motion.

3. The method of claim 1, wherein the fractional treatment is delivered while the handpiece is not in motion.

4. The method of claim 1, wherein the sensing comprises sensing the at least one positional parameter, and the automatically adjusting comprises automatically adjusting treatment density of the fractional treatment in real-time in response to the sensed at least one positional parameter.

5. The method of claim 4, wherein the order of steps in the method comprises:
1) manually moving a handpiece across a target region of skin;
2) sensing a positional parameter of the handpiece;
3) directing electromagnetic energy via the handpiece toward the target region in a manner so as to provide a fractional treatment; and
4) automatically adjusting treatment density of the fractional treatment in real-time in response to the sensed positional parameter.

6. The method of claim 1, wherein the sensing comprises sensing the at least one skin characteristic, and the automatically adjusting comprises automatically adjusting treatment density of the fractional treatment in real-time in response to the sensed at least one skin characteristic.

7. The method of claim 6, wherein the order of the steps in the method comprises:
1) manually moving a handpiece across a target region of skin;
2) sensing a skin characteristic of the target region;
3) automatically determining an appropriate treatment density of a fractional treatment in real-time in response to the sensed skin characteristic; and
4) directing electromagnetic energy via the handpiece toward the target region in a manner so as to provide the fractional treatment of the appropriate treatment density.

8. The method of claim 1, wherein the sensing comprises sensing the at least one skin response, and the automatically adjusting comprises automatically adjusting treatment density of the fractional treatment in real-time in response to the sensed at least one skin response.

9. The method of claim 8, wherein the order of the steps in the method comprises:
1) manually moving a handpiece across a target region of skin;
2) directing electromagnetic energy via the handpiece toward the target region in a manner so as to provide a fractional treatment;
3) sensing a skin response to the fractional treatment in the target region; and
automatically adjusting treatment density of the fractional treatment in real-time in response to the sensed skin response.

10. The method of claim 6, wherein said sensing a skin characteristic comprises sensing a skin response in the target region prior to and/or following the fractional treatment or portion thereof.

11. The method of claim 6, wherein said sensing a skin characteristic comprises generating, capturing and quantitatively comparing data corresponding to the skin characteristic prior to and/or following the fractional treatment or portion thereof.

12. The method of claim 6, wherein said sensing a skin characteristic comprises measuring the skin characteristic prior to at least one pulse of a treatment energy, and said automatically adjusting comprises automatically adjusting said treatment density for at least one pulse of the treatment energy.

13. The method of claim 6, wherein said sensing a skin characteristic comprises sensing skin birefringence prior to delivery of the fractional treatment or portion thereof.

14. The method of claim 6, wherein said sensing a skin characteristic comprises measuring skin tension prior to delivery of the fractional treatment or portion thereof.

15. The method of claim 6, wherein said sensing a skin characteristic comprises capturing images of the target region when illuminated with light of different polarizations prior to delivery of the fractional treatment or portion thereof.

16. The method of claim 6, wherein sensing a skin characteristic comprises capturing images of the target region when illuminated with light of different wavelength ranges prior to delivery of the fractional treatment or portion thereof.

17. The method of claim 6, wherein sensing a skin characteristic comprises measuring a change in distance between two features located in or on the target portion of skin.

18. The method of claim 6, wherein sensing a skin characteristic comprises sensing a blood vessel in the target portion of skin.

19. The method of claim 6, wherein sensing a skin characteristic comprises sensing a level of pigmentation in the target portion of skin.

20. The method of claim 8, wherein said automatically adjusting comprises adjusting density of the treatment zones if a skin response indicative of over-treatment or under-treatment is sensed.

21. The method of claim 8, wherein said sensing a skin response comprises sensing a skin response in the target region prior to the fractional treatment or portion thereof and following the fractional treatment or portion thereof.

22. The method of claim 8, wherein said sensing a skin response comprises generating, capturing and quantitatively comparing data corresponding to a skin condition prior to the fractional treatment or portion thereof and following the fractional treatment or portion thereof.

23. The method of claim 8, wherein said sensing a skin response comprises measuring the response to at least one prior pulse of a treatment energy, and said automatically adjusting comprises automatically adjusting said treatment density for at least one subsequent pulse of the treatment energy.

24. The method of claim 8, wherein said sensing a skin response comprises sensing changes in skin birefringence due to the fractional treatment or portion thereof.

25. The method of claim 8, wherein said sensing a skin response comprises measuring changes in skin tension due to the fractional treatment or portion thereof.

26. The method of claim 8, wherein said sensing a skin response comprises capturing images of the target region when illuminated with light of different polarizations following the fractional treatment or portion thereof.

27. The method of claim 8, wherein sensing a skin response comprises capturing images of the target region when illuminated with light of different wavelength ranges following the fractional treatment or portion thereof.

28. The method of claim 1, wherein the method further comprises automatically adjusting number of said treatment zones in real-time in response to said sensing.

29. The method of claim 1, wherein the method further comprises automatically adjusting size of said treatment zones in real time in response to said sensing.

30. The method of claim 1, wherein the method further comprises automatically adjusting spacing of said treatment zones in real time in response to said sensing.

31. An apparatus for controlled fractional skin treatment comprising:
at least one electromagnetic source configured to generate electromagnetic treatment energy capable of treating skin when delivered in a fractional manner;
a manually movable handpiece operably coupled to the electromagnetic source, wherein the handpiece is configured to deliver the electromagnetic treatment energy in a fractional manner to a target region of skin so as to provide a fractional skin treatment;
a controller operably coupled to the electromagnetic source, wherein the controller is configured to activate or adjust one or more parameters of the electromagnetic source; and
a sensor operably coupled to the controller, wherein the sensor is configured to sense at least one positional parameter, skin response and/or skin characteristic in the target region;
wherein positional, skin response and/or skin characteristic feedback from the sensor is used by the controller to control density of the fractional treatment by activation or adjustment of the one or more parameters of the electromagnetic source and/or handpiece.

32. The apparatus of claim 31, wherein the apparatus is configured to deliver the fractional skin treatment while the handpiece is in motion.

33. The apparatus of claim 31, wherein the apparatus is configured to deliver the fractional skin treatment while the handpiece is not in motion.

34. The apparatus of claim 31, wherein the apparatus further comprises a scanning delivery unit operably coupled to a scanner control that scans the electromagnetic treatment energy over the target region of the skin, and wherein the positional, skin response and/or skin characteristic feedback from the sensor is used by the controller to control the density of the fractional treatment by activation or adjustment of the one or more parameters of the electromagnetic source, handpiece, and/or scanner control.

35. The apparatus of claim 31, wherein the apparatus further comprises a contact plate mechanically coupled to the handpiece, wherein said contact plate is used to make good electrical or optical contact with the target region of skin to improve the operation of the sensor and/or to improve the delivery of the electromagnetic treatment energy.
